(19) United States
(12) Patent Application Publication
(10) Pub. No.: US 2023/0120273 A1
(43) Pub. Date: Apr. 20, 2023

(54) ACCELERATION OF MRI EXAMINATIONS

(71) Applicant: BAYER AKTIENGESELLSCHAFT, Leverkusen (DE)

(72) Inventors: Gesine KNOBLOCH, Berlin (DE); Christian LIENERTH, Frankfurt/Main (DE)

(21) Appl. No.: 17/754,559

(22) PCT Filed: Oct. 5, 2020
(86) PCT No.: PCT/EP2020/077775

§ 371 Date: Apr. 6, 2022

(30) Foreign Application Priority Data
Oct. 11, 2019 (EP) 19202711.8
May 6, 2020 (EP) 20173089.2

(51) Int. Cl. A61B 5/055; G01R 33/56

(52) U.S. Cl. CPC A61B 5/055; G01R 33/5601; G01R 33/281

(57) ABSTRACT

The disclosure relates to the acceleration of MRI examinations, particularly during the detection and differential diagnosis of focal liver lesions by way of dynamic contrast-enhanced magnetic resonance imaging (MRI). The disclosure also relates to a method, a system and a computer program product for generating MRI images, particularly of the liver.

The present disclosure deals with the quickening of MRI examinations, especially in the detection and differential diagnosis of focal liver lesions by means of dynamic contrast-enhancing magnetic resonance imaging (MRI). Subjects of the present disclosure are, in particular, a method, a system and a computer program product for generating MRI pictures, especially of the liver.

Magnetic resonance imaging, MRI for short, is an imaging method which is used especially in medical diagnostics for depicting structure and function of the tissues and organs in the human or animal body.

In MRI, the magnetic moments of protons in an examination object are aligned in a basic magnetic field, with the result that there is a macroscopic magnetization along a longitudinal direction. This is then deflected from the resting position by irradiation with high-frequency (HF) pulses (excitation). The return of the excited states to the resting position (relaxation), or magnetization dynamics, is then detected as relaxation signals by means of one or more HF receiver coils.

For spatial encoding, rapidly switched magnetic gradient fields are superimposed on the basic magnetic field. The captured relaxation signals, or detected and spatially resolved MRI data, are initially present as raw data in a spatial frequency space, and can be transformed by subsequent Fourier transform into real space (image space).

In the case of native MRI, the tissue contrasts are created by the different relaxation times (T1 and T2) and the proton density.

T1 relaxation describes the transition of the longitudinal magnetization to its equilibrium state, T1 being the time taken to reach 63.21% of the equilibrium magnetization prior to the resonance excitation. It is also called longitudinal relaxation time or spin-lattice relaxation time.

T2 relaxation describes in an analogous manner the transition of transverse magnetization to its equilibrium state.

MRI contrast agents exert their effect by altering the relaxation times of structures that take up contrast agents. A distinction can be made between two groups of substances: paramagnetic and superparamagnetic substances. Both groups of substances have unpaired electrons that induce a magnetic field around the individual atoms or molecules.

Superparamagnetic contrast agents result in a predominant shortening of T2, whereas paramagnetic contrast agents mainly lead to a shortening of T1. A shortening of the T1 time leads to an increase in the signal intensity in T1-weighted MRI images, and a shortening of the T2 time leads to a decrease in the signal intensity in T2-weighted MRI images.

The effect of said contrast agents is indirect, since the contrast agent itself does not emit a signal, but instead merely influences the intensity of signals in its vicinity.

An example of a superparamagnetic contrast agent is iron oxide nanoparticles (SPIO, superparamagnetic iron oxide).

Examples of paramagnetic contrast agents are gadolinium chelates such as gadopentetate dimeglumine (trade name: Magnevist® and others), gadobenate dimeglumine (trade name: Multihance®), gadoteric acid (Dotarem®, Dotagita®, Cyclolux®), gadodiamide (Omniscan®), gadoteridol (ProHance®) and gadobutrol (Gadovist®).

Extracellular, intracellular and intravascular contrast agents can be distinguished according to their pattern of spreading in the tissue.

Characteristic features of contrast agents based on gadoxetic acid are specific uptake by liver cells (hepatocytes), accumulation in the functional tissue (parenchyma), and enhancement of contrasts in healthy liver tissue. The cells of cysts, metastases, and most hepatocellular carcinomas no longer function in the same way as normal liver cells, show little or no uptake of contrast agent, are not depicted with enhancement, and are identifiable and localizable as a result.

Examples of contrast agents based on gadoxetic acid are described in U.S. Pat. No. 6,039,931A; they are commercially available for example under the trade names Primovist® or Eovist®.

The contrast-enhancing effect of Primovist®/Eovist® is mediated by the stable gadolinium complex Gd-EOB-DTPA (gadolinium ethoxybenzyl diethylenetriamine pentaacetic acid). DTPA forms with the paramagnetic gadolinium ion a complex that has extremely high thermodynamic stability. The ethoxybenzyl (EOB) radical is the mediator of the hepatobiliary uptake of the contrast agent.

Primovist® can be used for the detection of tumors in the liver. Blood supply to the healthy liver tissue is primarily achieved via the portal vein (vena portae), whereas the liver artery (arteria hepatica) supplies most primary tumors. After intravenous injection of a bolus of contrast agent, it is accordingly possible to observe a time delay between the signal rise of the healthy liver parenchyma and of the tumor.

Besides malignant tumors, what are commonly found in the liver are benign lesions such as cysts, haemangiomas and focal nodular hyperplasia (FNH). A proper planning of therapy requires that these be differentiated from the malignant tumors. Primovist® can be used for the identification of benign and malignant focal liver lesions. By means of T1-weighted MRI, it provides information about the character of said lesions. Differentiation is achieved by making use of the different blood supply to liver and tumor and of the temporal profile of contrast enhancement.

The contrast enhancement achieved by means of Primovist® can be divided into at least two phases: into a dynamic phase (comprising the so-called arterial phase, portal venous phase and late phase) and the hepatobiliary phase, in which a significant uptake of Primovist® into the hepatocytes has already taken place.

Tracking the spreading of contrast agent over time across the dynamic phase and the hepatobiliary phase provides a good way of detecting and differentially diagnosing focal liver lesions; however, the examination extends over a comparatively long time span. Over said time span, movements by the patient should be avoided in order to minimize movement artifacts in the MRI image. The lengthy restriction of movement can be unpleasant for a patient.

Proceeding from the described prior art, the technical object was that of reducing the MRI examination time span.

This object is achieved by the subjects of the independent claims. Preferred embodiments of the present disclosure are found in the dependent claims, in this description and in the drawings.

The present disclosure provides, in a first aspect, a method comprising the steps of:

receiving a first MRI image of an examination object, wherein the first MRI image shows a liver or part of a liver of the examination object at a first time point after an administration of a first contrast agent, wherein healthy liver cells are depicted with contrast enhancement in the first MRI image at the first time point as a consequence of the administration of the first contrast agent,

receiving a second MRI image of the same examination object, wherein the second MRI image shows the same liver or the same part of the liver at a second time point after an administration of a second contrast agent, wherein healthy liver cells are depicted with contrast enhancement in the second MRI image at the second time point as a consequence of the administration of the first contrast agent, and wherein blood vessels are depicted with contrast enhancement in the second MRI image at the second time point as a consequence of the administration of the second contrast agent,

generating a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image,

displaying and/or outputting the first MRI picture and/or storing the first MRI picture in a data storage medium.

The present disclosure further provides a system comprising:

a receiving unit,

a control and calculation unit, and

an output unit,

wherein the control and calculation unit is configured to prompt the receiving unit to receive a first MRI image of an examination object, wherein the first MRI image shows a liver or part of a liver of the examination object at a first time point after an administration of a first contrast agent, wherein healthy liver cells are depicted with contrast enhancement in the first MRI image at the first time point as a consequence of the administration of the first contrast agent,

wherein the control and calculation unit is configured to prompt the receiving unit to receive a second MRI image of an examination object, wherein the second MRI image shows the same liver or the same part of the liver at a second time point after an administration of a second contrast agent, wherein healthy liver cells are depicted with contrast enhancement in the second MRI image at the second time point as a consequence of the administration of the first contrast agent, and wherein blood vessels are depicted with contrast enhancement in the second MRI image at the second time point as a consequence of the administration of the second contrast agent,

wherein the control and calculation unit is configured to generate a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image,

wherein the control and calculation unit is configured to prompt the output unit to display the first MRI picture, to output it or to store it in a data storage medium.

The present disclosure further provides a computer program product comprising a computer program which can be loaded into a memory of a computer system, where it prompts the computer system to execute the following steps:

receiving a first MRI image of an examination object, wherein the first MRI image shows a liver or part of a liver of the examination object at a first time point after an administration of a first contrast agent, wherein healthy liver cells are depicted with contrast enhancement in the first MRI image at the first time point as a consequence of the administration of the first contrast agent,

receiving a second MRI image of the same examination object, wherein the second MRI image shows the same liver or the same part of the liver at a second time point after an administration of a second contrast agent, wherein healthy liver cells are depicted with contrast enhancement in the second MRI image at the second time point as a consequence of the administration of the first contrast agent, and wherein blood vessels are depicted with contrast enhancement in the second MRI image at the second time point as a consequence of the administration of the second contrast agent,

generating a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image,

displaying and/or outputting the first MRI picture and/or storing the first MRI picture in a data storage medium.

The present disclosure further provides for the use of a first and a second contrast agent in an MRI method, wherein the MRI method comprises the following steps:

administering the first contrast agent,

generating a first MRI image, wherein the first MRI image shows a liver or part of a liver at a first time point after the administration of the first contrast agent, wherein healthy liver cells are depicted with contrast enhancement in the first MRI image at the first time point as a consequence of the administration of the first contrast agent,

administering the second contrast agent at a second time point after the generation of the first MRI image,

generating a second MRI image, wherein the second MRI image shows the same liver or the same part of the liver at a second time point after the administration of the second contrast agent, wherein healthy liver cells are depicted with contrast enhancement in the second MRI image at the second time point as a consequence of the administration of the first contrast agent, and wherein blood vessels are depicted with contrast enhancement in the second MRI image at the second time point as a consequence of the administration of the second contrast agent,

generating a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image,

displaying and/or outputting the first MRI picture and/or storing the first MRI picture in a data storage medium.

Further provided is a first and a second contrast agent for use in an MRI method, wherein the MRI method comprises the following steps:

administering the first contrast agent,

generating a first MRI image, wherein the first MRI image shows a liver or part of a liver at a first time point after the administration of the first contrast agent, wherein healthy liver cells are depicted with contrast enhancement in the first MRI image at the first time point as a consequence of the administration of the first contrast agent,

administering the second contrast agent at a second time point after the generation of the first MRI image,

generating a second MRI image, wherein the second MRI image shows the same liver or the same part of the liver at a second time point after the administration of the second contrast agent, wherein healthy liver cells are depicted with contrast enhancement in the second MRI image at the second time point as a consequence of the administration of the first contrast agent, and wherein blood vessels are depicted with contrast enhancement in the second MRI image at the second time point as a consequence of the administration of the second contrast agent,

generating a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image,

displaying and/or outputting the first MRI picture and/or storing the first MRI picture in a data storage medium.

Further provided is a kit comprising a contrast agent and a computer program product according to the disclosure.

The disclosure will be more particularly elucidated below without distinguishing between the subjects of the disclosure (method, system, computer program product, use, contrast agent for use, kit). On the contrary, the following elucidations are intended to apply analogously to all the subjects of the disclosure, irrespective of in which context (method, system, computer program product, use, contrast agent for use, kit) they occur.

In the case of a classic MRI examination of the liver or part of the liver of an examination object, a hepatobiliary contrast agent is administered in the form of a single bolus. This is followed by the generation of a series of MRI images showing the liver or the part of the liver during the arterial phase, the portal venous phase, the late phase and the hepatobiliary phase. Usually, the examination object is already situated in the MRI scanner when administering the contrast agent. Since the contrast agent spreads comparatively slowly in the examination region after the intravenous administration in the form of a bolus, the MRI examination extends over a comparatively long period. About ten to twenty minutes are usual.

According to the disclosure, this examination period is shortened by administration of contrast agent in the form of two boluses, the examination object not yet being situated in the MRI scanner at the time point of the first administration.

A first contrast agent is administered as a bolus while the examination object is not yet situated in the MRI scanner, and a second contrast agent is administered as a bolus when the examination object is situated in the MRI scanner. The first contrast agent is a hepatobiliary contrast agent; the second contrast agent can be the same contrast agent as the first contrast agent; however, the second contrast agent can also be a different contrast agent, especially an extracellular contrast agent.

The MRI examination only starts at a time point in a (first) hepatobiliary phase, at which time point the first contrast agent is already leading to a contrast enhancement of healthy liver tissue. At said time point, which is also referred to in this description as first time point, a first MRI image is generated. Only at this time point is it necessary for the examination object to be situated in the MRI scanner. At said first time point, the arterial phase, the portal venous phase and the late phase as a consequence of the administration of the first contrast agent have already passed; therefore, it was not possible to generate MRI images showing the liver or the part of the liver during said arterial phase, said portal venous phase and/or said late phase. To nevertheless obtain MRI images of said phases, a second contrast agent is administered and the spreading of the second contrast agent via the blood vessels is tracked by means of MRI. In the case of the MRI images generated after the administration of the second contrast agent, there is still first contrast agent present in the healthy liver cells, meaning that the contrast between the blood vessels and the healthy liver cells is low. To increase the contrast between the healthy liver cells and the blood vessels, artificial MRI pictures having an increased contrast are generated from the MRI images after the administration of the second contrast agent using one or more MRI images before the administration of the second contrast agent but after the administration of the first contrast agent. In one embodiment, this is done by subtraction of an MRI image which was generated during the hepatobiliary phase after the administration of the first contrast agent and before the administration of the second contrast agent from one or more MRI images which were generated during the dynamic phase (i.e. during the arterial phase, the portal venous phase and/or the late phase) after the administration of the second contrast agent. In a further embodiment, artificial MRI pictures are generated with the aid of an artificial neural network. In both embodiments, artificial MRI pictures giving the impression that no first contrast agent had been administered, but only the second contrast agent had been administered, can be generated.

The “examination object” is usually a living being, preferably a mammal, very particularly preferably a human.

Part of the examination object is subjected to a contrast-enhanced magnetic resonance imaging examination. The “examination region”, also called image volume (field of view, FOV), is in particular a volume which is imaged in the magnetic resonance images. The examination region is typically defined by a radiologist, for example on an overview image (localizer). It is of course also possible for the examination region to alternatively or additionally be defined automatically, for example on the basis of a selected protocol. The examination region comprises at least part of the liver of the examination object.

A first contrast agent which spreads in the examination region is administered to the examination object. The first contrast agent is preferably administered intravenously as a bolus using dosing based on body weight (e.g. into an arm vein).

A “contrast agent” is understood to mean a substance or substance mixture, the presence of which in a magnetic resonance measurement leads to an altered signal. Preferably, the first contrast agent leads to a shortening of the T1 relaxation time and/or the T2 relaxation time.

The first contrast agent is a hepatobiliary contrast agent such as, for example, Gd-EOB-DTPA or Gd-BOPTA.

In a particularly preferred embodiment, the first contrast agent is a substance or a substance mixture having gadoxetic acid or a salt of gadoxetic acid as contrast-enhancing active substance. Very particular preference is given to the disodium salt of gadoxetic acid (Gd-EOB-DTPA disodium).

After the intravenous administration of the hepatobiliary contrast agent in the form of a bolus into an arm vein, the contrast agent reaches the liver first via the arteries. These are depicted with contrast enhancement in the corresponding MRI images. The phase in which the liver arteries are depicted with contrast enhancement in MRI images is referred to as “arterial phase”. Said phase starts immediately after the administration of the contrast agent and usually lasts 15 to 60 seconds.

Subsequently, the contrast agent reaches the liver via the liver veins. Whereas the contrast in the liver arteries is already decreasing, the contrast in the liver veins is reaching a maximum. The phase in which the liver veins are depicted with contrast enhancement in MRI images is referred to as “portal venous phase”. Said phase can already start during the arterial phase and overlap therewith. Usually, said phase starts 60 to 70 seconds after the intravenous administration and usually lasts 50 to 70 seconds.

The portal venous phase is followed by the “late phase”, in which the contrast in the liver arteries drops further and the contrast in the liver veins likewise drops. When using a hepatobiliary contrast agent, the contrast in the healthy liver cells gradually rises in the late phase. Said phase usually starts 100 to 140 seconds after the administration of the contrast agent and usually lasts 50 to 70 seconds.

The arterial phase, the portal venous phase and the late phase are also referred to collectively as “dynamic phase”.

10-20 minutes after its injection, a hepatobiliary contrast agent leads to a distinct signal enhancement in the healthy liver parenchyma. This phase is referred to as “hepatobiliary phase”. The contrast agent is eliminated only slowly from the liver cells; accordingly, the hepatobiliary phase can last for two hours and longer.

The stated phases are, for example, described in more detail in the following publications: J. Magn. Reson. Imaging, 2012, 35(3): 492-511, doi:10.1002/jmri.22833; Clujul Medical, 2015, Vol. 88 no. 4: 438-448, DOI: 10.15386/cjmed-414; Journal of Hepatology, 2019, Vol. 71: 534-542, (http://dx.doi.org/10.1016/j.jhep.2019.05.005).

An extracellular contrast agent is not taken up by the liver cells and does not accumulate in the healthy liver tissue. An extracellular contrast agent therefore exhibits only a dynamic phase (comprising an arterial phase, a portal venous phase and a late phase).

According to the disclosure, contrast agent is administered in the form of two boluses. The first administration is done at a time point at which the examination object is not yet situated in the MRI scanner. In the case of the first administration, the first contrast agent is administered.

After the administration of the first contrast agent, a time span can be waited for before the examination object is introduced into the MRI scanner and a first MRI image is generated at a first time point. The time span between the first administration and the generation of the first MRI image is preferably within the range from 5 minutes to 1 hour, yet more preferably within the range from 10 minutes to 30 minutes, and most preferably within the range from 8 minutes to 25 minutes.

The first MRI image shows the liver or part of the liver of the examination object during the hepatobiliary phase after the administration of the first contrast agent. Healthy liver cells are depicted with contrast enhancement in the first MRI image as a consequence of the administration of the first contrast agent.

The hepatobiliary phase in which the first MRI image is generated is also referred to as first hepatobiliary phase in this description. The first contrast agent has reached the healthy liver cells and leads to contrast enhancement, to signal enhancement of the healthy liver cells in the case of a paramagnetic contrast agent. During the arterial phase, the portal venous phase and the late phase which occur after the administration of the first contrast agent, no MRI images are generated. The arterial phase, the portal venous phase and the late phase which occur after the administration of the first contrast agent are also referred to as first arterial phase, first portal venous phase and first late phase in this description.

It is conceivable that multiple MRI images are generated during the first hepatobiliary phase.

After the generation of one or more first MRI images during the first hepatobiliary phase, contrast agent is administered a second time. What is administered the second time is a second contrast agent. The second contrast agent can be the same contrast agent as the first contrast agent; however, the second contrast agent can also be a different contrast agent, preferably an extracellular one. The second contrast agent is likewise preferably administered intravenously as a bolus using dosing based on body weight (e.g. into an arm vein).

The administration of the first contrast agent is also referred to as first administration in this description; the administration of the second contrast agent is also referred to as second administration in this description. If the first contrast agent and the second contrast agent are identical, then what thus takes place is a first administration of a hepatobiliary contrast agent and, at a later time point, a second administration of the hepatobiliary contrast agent. If the first contrast agent and the second contrast agent are different, what takes place is a first administration of a first contrast agent, said first contrast agent being a hepatobiliary contrast agent, and what takes place at a later time point is a second administration of a second (different) contrast agent.

At the time point of the second administration (or at the time point of the administration of the second contrast agent), the examination object is preferably already situated in the MRI scanner. After the administration of the second contrast agent, an arterial phase, a portal venous phase and a late phase is again passed through. Said arterial phase, portal venous phase and late phase are also referred to as second arterial phase, second portal venous phase and second late phase in this description. In the second arterial phase and/or in the second portal venous phase and/or in the second late phase, an MRI image or multiple MRI images is/are generated. Said MRI images are referred to in the order of their acquisition as second, third, fourth, etc.

In a preferred embodiment, a second MRI image is generated during the second arterial phase, a third MRI image is generated during the second portal venous phase and a fourth MRI image is generated during the second late phase. Such a second MRI image shows especially arteries with contrast enhancement; such a third MRI image shows especially veins with contrast enhancement.

It is further conceivable that more than one MRI image is generated during the stated phases.

The measured MRI images can be present as two-dimensional images showing a sectional plane through the examination object. The measured MRI images can be present as a stack of two-dimensional images, with each individual image of the stack showing a different sectional plane. The measured MRI images can be present as three-dimensional images (3D images). In the interests of simpler illustration, the disclosure will be elucidated at some points in the present description on the basis of the presence of two-dimensional MRI images, without any wish, however, to restrict the disclosure to two-dimensional MRI images. It is clear to a person skilled in the art how it is possible to apply what is respectively described to stacks of two-dimensional images and to 3D images (see, in relation to this, for example M. Reisler, W. Semmler Magnetresonanztomographie [Magnetic resonance imaging], Springer Verlag, 3rd edition, 2002, ISBN: 978-3-642-63076-7).

Usually, the measured MRI images are present as digital image files. The term “digital” means that the MRI images can be processed by a machine, generally a computer system. “Processing” is understood to mean the known methods for electronic data processing (EDP).

Digital image files can be present in various formats. For example, digital image files can be coded as raster graphics. Raster graphics consist of a grid arrangement of so-called picture elements (pixel) or volume elements (voxel), to which a color or a gray value is assigned in each case. The main features of a 2D raster graphic are therefore the image size (width and height measured in pixels, also informally called image resolution) and the color depth. A color is usually assigned to a picture element of a digital image file. The color coding used for a picture element is defined, inter alia, in terms of the color space and the color depth. The simplest case is a binary image, in which a picture element stores a black-and-white value. In the case of an image, the color of which is defined in terms of the so-called RGB color space (RGB stands for the primary colors red, green and blue), each picture element consists of three subpixels, a subpixel for the color red, a subpixel for the color green and a subpixel for the color blue. The color of a picture element arises through the superimposition (additive blending) of the color values of the subpixels. The color value of a subpixel is divided into 256 color nuances, which are called tonal values and usually range from 0 to 255. The color nuance “0” of each color channel is the darkest. If all three channels have the tonal value 0, the corresponding picture element appears black; if all three channels have the tonal value 255, the corresponding picture element appears white. When carrying out the present disclosure, digital image files (MRI images) are subjected to certain operations. In this connection, the operations affect predominantly the picture elements, or the tonal values of the individual picture elements. There are a multiplicity of possible digital image formats and color codings. For simplification, it is assumed in this description that the present images are gray-scale raster graphics having a specific number of picture elements, with each picture element being assigned a tonal value indicating the gray value of the image. However, this assumption is not in any way to be understood as limiting. It is clear to a person skilled in the art of image processing how the teaching of said description can be applied to image files which are present in other image formats and/or in which the color values are coded differently.

In a next step, MRI pictures are generated from the MRI images which were generated during one or more phases after the administration of the first and the second contrast agent.

The term “MRI picture” is a coinage. The term has been created in order to be able to better distinguish between the various MRI images generated and used in the context of the disclosure. Whereas a first, second, third and fourth MRI image in the context of the present disclosure is an image generated by measurement, an MRI picture is understood to mean an artificial depiction that is the result of calculations. Thus, an MRI picture itself is not generated by measurement; instead, it is calculated from at least two MRI images generated by measurement. MRI pictures are preferably in the same form as the MRI images from which they are generated (e.g. in the form of digital image files).

Preferably using the first MRI image, what is calculated from each MRI image which was generated during one or more phases after the administration of the second contrast agent is an MRI picture in each case: what is calculated from the second MRI image and preferably the first MRI image is a first MRI picture; if present, what is calculated from the third MRI image and preferably the first MRI image is a second MRI picture; if present, what is calculated from the fourth MRI image and preferably the first MRI image is a third MRI picture; and so forth.

The goal of generating MRI pictures from the MRI images is to increase the contrast between healthy liver tissue and other regions. When using a hepatobiliary paramagnetic contrast agent as the first contrast agent, the signal intensity of healthy liver tissue during the second arterial phase, the second portal venous phase and the second late phase is still elevated as a consequence of the administration of the first contrast agent. The second contrast agent which spreads in the stated second phases likewise leads to an elevated signal in the tissue in which it spreads. This means that there is only a low contrast in the MRI images between the healthy liver tissue and the remaining tissue, which remaining tissue is contrast-enhanced due to (second) contrast agent. In a preferred embodiment, said contrast is increased by subtracting the first MRI image from the second MRI image. If present, the first MRI image can also be subtracted from the third MRI image. If present, the first MRI image can also be subtracted from the fourth MRI image. And so on.

In the case of such a subtraction, the tonal values of corresponding pixels or voxels are usually subtracted from one another. Two pixels or voxels correspond to one another when they show the same examination region.

It is conceivable that the MRI images subjected to a subtraction are subjected to a movement correction beforehand. Such a movement correction ensures that a pixel or voxel of the first MRI image shows the same examination region as the corresponding pixel or voxel of the second MRI image, the third MRI image, the fourth MRI image and so on. Movement correction methods are described in the prior art (see, for example EP3118644, EP3322997, US20080317315, US20170269182, US20140062481, EP2626718).

The subtraction of MRI images reduces in particular the signal intensity in the healthy liver tissue.

The MRI pictures generated in the subtraction of the first MRI image from the second MRI image and from the third MRI image and from the fourth MRI image, and so on, show the spreading of contrast agent in the tissue approximately as it would be if only contrast agent in the form of a single bolus had been administered—with the difference that the MRI examination is shortened, since the examination object has to occupy a shorter time span in the MRI scanner.

In a further embodiment, one or more MRI pictures are calculated with the aid of an artificial neural network which has been trained to generate an MRI picture from a first MRI image and a second MRI image. The first MRI image shows a liver or part of a liver of an examination object in a first hepatobiliary phase, i.e. at a first time point after an administration of a hepatobiliary contrast agent, wherein healthy liver cells are depicted with contrast enhancement in the first MRI image at the first time point as a consequence of the administration of the first contrast agent, The second MRI image shows the same liver or the same part of the liver at a second time point after an administration of a second contrast agent, wherein healthy liver cells are depicted with contrast enhancement in the second

MRI image at the second time point as a consequence of the administration of the first contrast agent, and wherein blood vessels are depicted with contrast enhancement in the second MRI image at the second time point as a consequence of the administration of the second contrast agent. The MRI picture shows the same liver or the same part of the liver as it would appear if only the second contrast agent had been administered: the blood vessels are depicted with contrast enhancement as a consequence of the administration of the second contrast agent, but healthy liver cells are not depicted with contrast enhancement as a consequence of the administration of a first contrast agent. In other words, what is generated is an MRI picture which looks like the second MRI image, with the difference that the contrast enhancement of the healthy liver cells, which was caused by the administration of the first contrast agent, is subtracted (eliminated) from the second MRI image.

Such an artificial neural network comprises at least three layers of processing elements: a first layer with input neurons (nodes), an N-th layer with at least one output neuron (nodes) and N-2 inner layers, where N is a natural number and greater than 2.

The input neurons serve to receive digital MRI images as input values, for example a first MRI image and a second MRI image. Normally, there is one input neuron for each pixel or voxel of a digital MRI image. There can be additional input neurons for additional input values (e.g. information about the examination region, about the examination object, about conditions which prevailed when generating the MRI images, and/or information about the time points at which or time spans in which the MRI images were generated).

In such a network, the output neurons serve to output an MRI picture.

The processing elements of the layers between the input neurons and the output neurons are connected to one another in a predetermined pattern with predetermined connection weights.

Preferably, the artificial neural network is a so-called convolutional neural network (CNN for short).

A convolutional neural network is capable of processing input data in the form of a matrix. This makes it possible to use digital MRI images represented as a matrix (e.g. width x height x color channels) as input data. By contrast, a normal neural network, for example in the form of a multilayer perceptron (MLP), requires a vector as input, i.e. to use an MRI image as input, the pixels or voxels of the MRI image would have to be rolled out successively in a long chain As a result, normal neural networks are, for example, not capable of recognizing objects in an MRI image independently of the position of the object in the MRI image. The same object at a different position in the MRI image would have a completely different input vector.

A CNN consists essentially of filters (convolutional layer) and aggregation layers (pooling layer) which are repeated alternately and, at the end, of one layer or multiple layers of “normal” completely connected neurons (dense/fully connected layer).

Details can be gathered from the prior art (see, for example: S. Khan et al.: A Guide to Convolutional Neural Networks for Computer Vision, Morgan & Claypool Publishers 2018, ISBN 1681730227, 9781681730226, WO2018/183044A1, WO2018/200493, WO2019/074938A1, WO2019/204406A1, WO2019/241659A1).

The neural network can be trained in a supervised machine learning process with a training data set. The training data set comprises a multiplicity of first reference MRI images, second reference MRI images and third reference MRI images.

Such a first reference MRI image corresponds to a first MRI image: it shows a liver or part of a liver of an examination object at a first time point after an administration of a first contrast agent, wherein healthy liver cells are depicted with contrast enhancement in the first MRI image at the first time point as a consequence of the administration of the first contrast agent.

Such a second reference MRI image corresponds to a second MRI image: it shows the same liver or the same part of the liver at a second time point after an administration of a second contrast agent, wherein healthy liver cells are depicted with contrast enhancement in the second MRI image at the second time point as a consequence of the administration of the first contrast agent, and wherein blood vessels are depicted with contrast enhancement in the second MRI image at the second time point as a consequence of the administration of the second contrast agent. The blood vessels can be arteries and/or veins.

Such a third reference MRI image shows the same liver or the same part of the liver at a second time point after an administration of a second contrast agent without a first contrast agent having been administered before the second time point: only blood vessels are depicted with contrast enhancement in the second MRI image at the second time point as a consequence of the administration of the second contrast agent.

During machine learning, a statistical model based on the training data is generated. This means that the examples are not simply learnt by heart, but that the model “recognizes” patterns and regularities in the training data. The model can thus also assess unknown data. Validation data can be used to test the quality of the assessment of unknown data.

The model is usually trained by means of supervised learning, i.e. first and second reference MRI images are presented to the model and it is informed of which third reference MRI images are associated with the particular first and second reference MRI images. The algorithm then learns a relationship between the reference MRI images in order to predict (to calculate) third MRI pictures for unknown first and second MRI images.

Self-learning algorithms trained by means of supervised learning are widely described in the prior art (see, for example, C. Perez: Machine Learning Techniques: Supervised Learning and Classification, Amazon Digital Services LLC—Kdp Print Us, 2019, ISBN 1096996545, 9781096996545, WO2018/183044A1, WO2018/200493, WO2019/074938A1, WO2019/204406A1, WO2019/241659A1).

The training of the neural network can, for example, be carried out by means of a backpropagation method. The aim here in respect of the network is maximum reliability of mapping of given input data onto given output data. The mapping quality is described by an error function. The goal is to minimize the error function. In the case of the backpropagation method, an artificial neural network is taught by the alteration of the connection weights.

In the trained state, the connection weights between the processing elements contain information regarding the relationship between the first and second reference MRI images and the third reference MRI images that can be used in order to predict the corresponding MRI pictures for new first and second MRI images.

A cross-validation method can be used in order to divide the data into training and validation data sets. The training data set is used in the backpropagation training of network weights. The validation data set is used in order to check the accuracy of prediction with which the trained network can be applied to unknown MRI images.

As already described, further information about the examination object, about the examination region, about examination conditions and/or about the measured MRI images can also be used for training, validation and prediction.

Examples of information about the examination object are: sex, age, weight, height, anamnesis, nature and duration and amount of medicaments already ingested, blood pressure, central venous pressure, breathing rate, serum albumin, total bilirubin, blood sugar, iron content, breathing capacity and the like. These can, for example, also be gathered from a database or an electronic patient file.

Examples of information about the examination region are: pre-existing conditions, operations, partial resection, liver transplantation, iron liver, fatty liver and the like.

It is conceivable that MRI images are subjected to a movement correction before they are fed to the prediction model. Such a movement correction ensures that a pixel or voxel of a first MRI image shows the same examination region as the corresponding pixel or voxel of a second MRI image. Movement correction methods are described in the prior art (see, for example: EP3118644, EP3322997, US20080317315, US20170269182, US20140062481, EP2626718).

The present disclosure provides a system which makes it possible to execute the method according to the disclosure.

Such a system comprises a receiving unit, a control and calculation unit and an output unit.

It is conceivable that said units are components of a single computer system, but it is also conceivable that said units are components of a plurality of separate computer systems that are connected to one another via a network in order to transmit data and/or control signals from one unit to another unit.

A “computer system” is an electronic data processing system that processes data by way of programmable computing rules. Such a system usually comprises a “computer”, the unit that comprises a processor for carrying out logical operations, and also a peripheral.

In computer technology, “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

Modern computer systems are frequently divided into desktop PCs, portable PCs, laptops, notebooks, netbooks and tablet PCs, and what are called handhelds (for example smartphones); all of these systems may be used to implement the disclosure.

Inputs into the computer system are achieved via input means such as, for example, a keyboard, a mouse, a microphone, a touch-sensitive display and/or the like.

The system according to the disclosure is configured to generate one MRI picture in each case from two MRI images at a time. Preferably, this is done by subtraction of the tonal values of corresponding picture elements of the two MRI images. In this connection, it is conceivable that the absolute difference is formed in order to avoid negative tonal values. Furthermore, it is conceivable that a negative tonal value is set to the tonal value zero in order to avoid negative tonal values.

The receiving unit serves for the receiving of MRI images. The MRI images can, for example, be transmitted from a magnetic resonance system or be read from a data storage medium. The magnetic resonance system may be a component of the system according to the disclosure. However, it is also conceivable that the system according to the disclosure is a component of a magnetic resonance system.

At least the first MRI image and the second MRI image are transmitted from the receiving unit to the control and calculation unit. If present, the third, fourth—and so forth—MRI images can also be transmitted.

The control and calculation unit serves for the calculation of MRI pictures from the received MRI images. Furthermore, the control and calculation unit serves for the control of the receiving unit and for the coordination of the data and signal flows between various units. It is conceivable that multiple control and calculation units are present.

Via the output unit, the calculated MRI pictures can be displayed (e.g. on a monitor), be outputted (e.g. via a printer) or be stored in a data storage medium.

Further embodiments of the disclosure are:

1. A method comprising the steps of:

receiving a first MRI image of an examination object, wherein the first MRI image shows a liver or part of a liver of the examination object at a first time point after a first administration of a contrast agent, wherein healthy liver cells are depicted with contrast enhancement in the first MRI image at the first time point as a consequence of the first administration of the contrast agent,

receiving a second MRI image of the same examination object, wherein the second MRI image shows the same liver or the same part of the liver at a second time point after a second administration of the contrast agent, wherein healthy liver cells are depicted with contrast enhancement in the second MRI image at the second time point as a consequence of the first administration of the contrast agent, and wherein blood vessels are depicted with contrast enhancement in the second MRI image at the second time point as a consequence of the second administration of the contrast agent,

generating a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image,

displaying and/or outputting the first MRI picture and/or storing the first MRI picture in a data storage medium.

2. The method according to embodiment 1, comprising the steps of:

receiving the first MRI image of the examination object, wherein the first MRI image shows the liver or the part of a liver of the examination object during a first hepatobiliary phase at the first time point after the first administration of the contrast agent,

receiving the second MRI image of the same examination object, wherein the second MRI image shows the same liver or the same part of the liver during a second arterial phase at the second time point after the second administration of the contrast agent,

receiving a third MRI image of the same examination object, wherein the third MRI image shows the same liver or the same part of the liver during a second portal venous phase at a third time point after the second administration of the contrast agent,

receiving a fourth MRI image of the same examination object, wherein the fourth MRI image shows the same liver or the same part of the liver during a second late phase at a fourth time point after the second administration of the contrast agent,

generating MRI pictures from the received MRI images, wherein the first MRI picture is calculated by subtraction of the first MRI image from the second MRI image, wherein a second MRI picture is calculated by subtraction of the first MRI image from the third MRI image, wherein a third MRI picture is calculated by subtraction of the first MRI image from the third MRI image,

displaying and/or outputting one or more of the generated MRI pictures and/or storing one or more of the generated MRI pictures in a data storage medium.

3. The method according to either of embodiments 1 and 2, wherein the MRI images are T1-weighted depictions of the liver or the part of the liver after administration of a hepatobiliary, paramagnetic contrast agent.

4. The method according to any of embodiments 1 to 3, wherein the contrast agent is a substance or a substance mixture having gadoxetic acid or a salt of gadoxetic acid as contrast-enhancing active substance, preferably the disodium salt of gadoxetic acid.

5. The method according to any of embodiments 1 to 4, wherein the examination object is a mammal, preferably a human.

6. A system comprising:

a receiving unit,

a control and calculation unit, and

an output unit,

wherein the control and calculation unit is configured to prompt the receiving unit to receive a first MRI image of an examination object, wherein the first MRI image shows a liver or part of a liver of the examination object at a first time point after a first administration of a contrast agent, wherein healthy liver cells are depicted with contrast enhancement in the first MRI image at the first time point as a consequence of the first administration of the contrast agent,

wherein the control and calculation unit is configured to prompt the receiving unit to receive a second MRI image of an examination object, wherein the second MRI image shows the same liver or the same part of the liver at a second time point after a second administration of the contrast agent, wherein healthy liver cells are depicted with contrast enhancement in the second MRI image at the second time point as a consequence of the first administration of the contrast agent, and wherein blood vessels are depicted with contrast enhancement in the second MRI image at the second time point as a consequence of the second administration of the contrast agent,

wherein the control and calculation unit is configured to generate a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image,

wherein the control and calculation unit is configured to prompt the output unit to display the first MRI picture, to output it or to store it in a data storage medium.

7. The computer system according to embodiment 6,

wherein the control and calculation unit is configured to prompt the receiving unit to receive a first MRI image, a second MRI image, a third MRI image and a fourth MRI image,
wherein the first MRI image shows the liver or the part of the liver of the examination object during a first hepatobiliary phase at the first time point after the first administration of the contrast agent,
wherein the second MRI image shows the same liver or the same part of the liver during a second arterial phase at a second time point after the second administration of the contrast agent,
wherein the third MRI image shows the same liver or the same part of the liver during a second portal venous phase at a third time point after the second administration of the contrast agent,
wherein the fourth MRI image shows the same liver or the same part of the liver during a second late phase at a fourth time point after the second administration of the contrast agent,

wherein the control and calculation unit is configured to calculate MRI pictures from the received MRI images, wherein a first MRI picture is calculated by subtraction of the first MRI image from the second MRI image, wherein a second MRI picture is calculated by subtraction of the first MRI image from the third MRI image, wherein a third MRI picture is calculated by subtraction of the first MRI image from the third MRI image,

wherein the control and calculation unit is configured to prompt the output unit to display one or more of the calculated MRI pictures on a screen and/or to output them on a printer and/or to store them in a data storage medium.

8. A computer program product comprising a computer program which can be loaded into a memory of a computer, where it prompts the computer to execute the following steps:

receiving a first MRI image of an examination object, wherein the first MRI image shows a liver or part of a liver of the examination object at a first time point after a first administration of a contrast agent, wherein healthy liver cells are depicted with contrast enhancement in the first MRI image at the first time point as a consequence of the first administration of the contrast agent,

receiving a second MRI image, wherein the second MRI image shows the same liver or the same part of the liver at a second time point after a second administration of the contrast agent, wherein healthy liver cells are depicted with contrast enhancement in the second MRI image at the second time point as a consequence of the first administration of the contrast agent, and wherein blood vessels are depicted with contrast enhancement in the second MRI image at the second time point as a consequence of the second administration of the contrast agent,

generating a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image,

displaying and/or outputting the first MRI picture and/or storing the first MRI picture in a data storage medium.

9. The computer program product according to embodiment 8, wherein the computer program prompts the computer to execute the following steps:

receiving the first MRI image of the examination object, wherein the first MRI image shows the liver or the part of the liver of the examination object during a first hepatobiliary phase at the first time point after the first administration of the contrast agent,

receiving the second MRI image, wherein the second MRI image shows the same liver or the same part of the liver during a second arterial phase at the second time point after the second administration of the contrast agent,

receiving a third MRI image, wherein the third MRI image shows the same liver or the same part of the liver during a second portal venous phase at a third time point after the second administration of the contrast agent,

receiving a fourth MRI image, wherein the fourth MRI image shows the same liver or the same part of the liver during a second late phase at a fourth time point after the second administration of the contrast agent,

generating MRI pictures from the received MRI images, wherein the first MRI picture is calculated by subtraction of the first MRI image from the second MRI image, wherein a second MRI picture is calculated by subtraction of the first MRI image from the third MRI image, wherein a third MRI picture is calculated by subtraction of the first MRI image from the third MRI image,

displaying and/or outputting one or more of the generated MRI pictures and/or storing one or more of the generated MRI pictures in a data storage medium.

10. The computer program product according to embodiment 9, wherein the computer program prompts the computer to execute one or more of the steps listed in claims 1 to 5.

11. Use of a contrast agent in an MRI method, wherein the MRI method comprises the following steps:

performing a first administration of the contrast agent,

generating a first MRI image, wherein the first MRI image shows a liver or part of a liver at a first time point after the first administration of the contrast agent, wherein healthy liver cells are depicted with contrast enhancement in the first MRI image at the first time point as a consequence of the first administration of the contrast agent,

performing a second administration of the contrast agent at a time point after the generation of the first MRI image,

generating a second MRI image, wherein the second MRI image shows the same liver or the same part of the liver at a second time point after the second administration of the contrast agent, wherein healthy liver cells are depicted with contrast enhancement in the second MRI image at the second time point as a consequence of the first administration of the contrast agent, and wherein blood vessels are depicted with contrast enhancement in the second MRI image at the second time point as a consequence of the second administration of the contrast agent,

generating a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image,

displaying and/or outputting one or more of the generated MRI pictures and/or storing one or more of the generated MRI pictures in a data storage medium.

12. The use according to embodiment 11, comprising the steps of:

performing a first administration of the contrast agent,

generating the first MRI image, wherein the first MRI image shows the liver or the part of the liver of the examination object during a first hepatobiliary phase at the first time point after the first administration of the contrast agent,

performing a second administration of the contrast agent at a time point after the generation of the first MRI image,

generating the second MRI image, wherein the second MRI image shows the same liver or the same part of the liver during a second arterial phase at the second time point after the second administration of the contrast agent,

generating a third MRI image, wherein the third MRI image shows the same liver or the same part of the liver during a second portal venous phase at a third time point after the second administration of the contrast agent,

generating a fourth MRI image, wherein the fourth MRI image shows the same liver or the same part of the liver during a second late phase at a fourth time point after the second administration of the contrast agent,

generating MRI pictures from the received MRI images, wherein the first MRI picture is calculated by subtraction of the first MRI image from the second MRI image, wherein a second MRI picture is calculated by subtraction of the first MRI image from the third MRI image, wherein a third MRI picture is calculated by subtraction of the first MRI image from the third MRI image,

displaying and/or outputting one or more of the generated MRI pictures and/or storing one or more of the generated MRI pictures in a data storage medium.

13. A contrast agent for use in an MRI method, wherein the MRI method comprises the following steps:

performing a first administration of the contrast agent,

generating a first MRI image, wherein the first MRI image shows a liver or part of a liver at a first time point after the first administration of the contrast agent, wherein healthy liver cells are depicted with contrast enhancement in the first MRI image at the first time point as a consequence of the first administration of the contrast agent,

performing a second administration of the contrast agent at a time point after the generation of the first MRI image,

generating a second MRI image, wherein the second MRI image shows the same liver or the same part of the liver at a second time point after the second administration of the contrast agent, wherein healthy liver cells are depicted with contrast enhancement in the second MRI image at the second time point as a consequence of the first administration of the contrast agent, and wherein blood vessels are depicted with contrast enhancement in the second MRI image at the second time point as a consequence of the second administration of the contrast agent,

generating a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image,

displaying and/or outputting one or more of the generated MRI pictures and/or storing one or more of the generated MRI pictures in a data storage medium.

14. A contrast agent for use according to embodiment 13, wherein the MRI method comprises the following steps:

performing a first administration of the contrast agent,

generating the first MRI image, wherein the first MRI image shows the liver or the part of the liver of the examination object during a first hepatobiliary phase at the first time point after the first administration of the contrast agent,

performing a second administration of the contrast agent at a time point after the generation of the first MRI image,

generating the second MRI image, wherein the second MRI image shows the same liver or the same part of the liver during a second arterial phase at the second time point after the second administration of the contrast agent,

generating a third MRI image, wherein the third MRI image shows the same liver or the same part of the liver during a second portal venous phase at a third time point after the second administration of the contrast agent,

generating a fourth MRI image, wherein the fourth MRI image shows the same liver or the same part of the liver during a second late phase at a fourth time point after the second administration of the contrast agent,

generating MRI pictures from the received MRI images, wherein the first MRI picture is calculated by subtraction of the first MRI image from the second MRI image, wherein a second MRI picture is calculated by subtraction of the first MRI image from the third MRI image, wherein a third MRI picture is calculated by subtraction of the first MRI image from the third MRI image,

displaying and/or outputting one or more of the generated MRI pictures and/or storing one or more of the generated MRI pictures in a data storage medium.

15. A contrast agent for use according to either of embodiments 13 and 14, wherein the contrast agent is a substance or a substance mixture having gadoxetic acid or a salt of gadoxetic acid as contrast-enhancing active substance, preferably the disodium salt of gadoxetic acid.

16. A kit comprising a contrast agent according to either of embodiments 13 and 14 and a computer program product according to any of embodiments 8, 9 and 10.

The disclosure is elucidated in detail hereinbelow with reference to figures, without any intention to restrict the disclosure to the features or combinations of features shown in the figures.

In the figures:

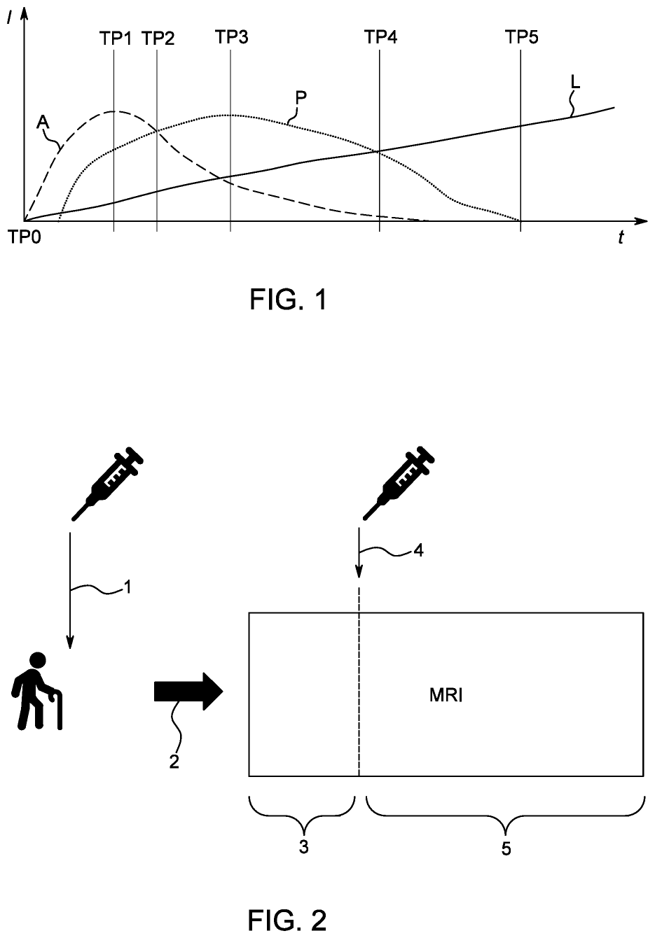

FIG. 1 shows schematically the temporal profile of the concentrations of contrast agent in the liver arteries (A), the liver veins (P) and the liver cells (L). The concentrations are depicted in the form of the signal intensities I in the stated areas (liver arteries, liver veins, liver cells) in the magnetic resonance measurement as a function of the time t. Upon an intravenous bolus injection, the concentration of the contrast agent rises in the liver arteries (A) first of all (dashed curve). The concentration passes through a maximum and then drops. The concentration in the liver veins (P) rises more slowly than in the liver arteries and reaches its maximum later (dotted curve). The concentration of the contrast agent in the liver cells rises slowly (solid curve) and reaches its maximum only at a very much later time point (not depicted in FIG. 1). A few characteristic time points can be defined: At time point TP0, contrast agent is administered intravenously as a bolus. At time point TP1, the concentration (the signal intensity) of the contrast agent in the liver arteries reaches its maximum. At time point TP2, the curves of the signal intensities for the liver arteries and the liver veins intersect. At time point TP3, the concentration (the signal intensity) of the contrast agent in the liver veins passes through its maximum. At time point TP4, the curves of the signal intensities for the liver arteries and the liver cells intersect. At time point T5, the concentrations in the liver arteries and the liver veins have dropped to a level at which they no longer cause a measurable contrast enhancement.

According to the disclosure, contrast agent is administered in the form of two boluses. A first bolus containing a first contrast agent is administered at time point TP0, and a second bolus containing a second contrast agent is administered at a time point after time point TP3, preferably after time point TP4, even more preferably after time point TP5. A first MRI image is preferably generated after time point TP4, even more preferably after time point TP5. After the administration of the second contrast agent, at least the arterial phase and the portal venous phase are passed through a second time. These phases are characterized by an analogous concentration profile of contrast agent in the arteries and the liver veins, i.e. the time points TP1 (maximum contrast enhancement of the arteries), TP2 (equal contrast enhancement of arteries and veins) and TP3 (maximum contrast enhancement of the veins) occur a second time; they can be referred to as time points TP1′, TP2′ and TP3′. Preferably, a second MRI image and possibly a third MRI image and possibly further MRI images are generated within a time span between the time point of administration of the second contrast agent and time point TP3′.

FIG. 2 shows schematically an example of an MRI examination shortened according to the disclosure. Here, a first contrast agent is first administered in the form of a first bolus (1). The examination object is passed to MRI after a certain waiting period (2), for example 8 to 20 minutes. Thereafter, the MRI process is started and a first MRI image (3) showing the liver of the examination object or part of said liver in the hepatobiliary phase is generated. Thereafter, a second contrast agent is administered intravenously in the form of a bolus injection (4) to the examination object and subsequently one or more further MRI images of the liver or part of said liver in the dynamic phase are generated.

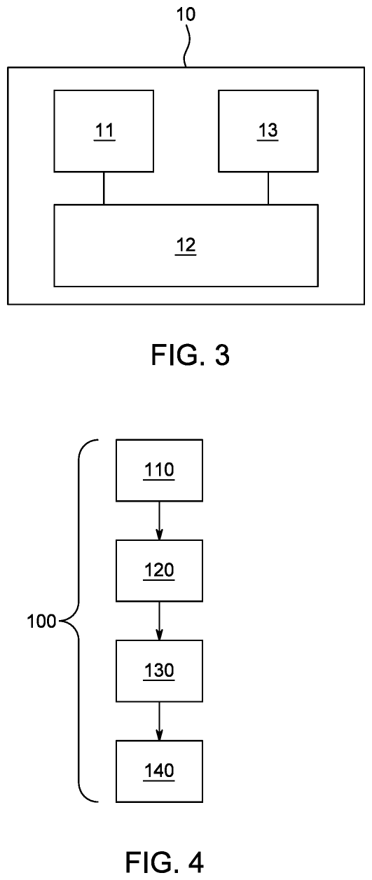

FIG. 3 shows schematically a preferred embodiment of the system according to the disclosure. The system (10) comprises a receiving unit (11), a control and calculation unit (12) and an output unit (13).

The control and calculation unit (12) is configured to prompt the receiving unit (11) to receive a first MRI image of an examination object, wherein the first MRI image shows a liver or part of a liver of the examination object at a first time point after an administration of a first contrast agent, wherein healthy liver cells are depicted with contrast enhancement in the first MRI image at the first time point as a consequence of the administration of the first contrast agent.

The control and calculation unit (12) is further configured to prompt the receiving unit (11) to receive a second MRI image of an examination object, wherein the second MRI image shows the same liver or the same part of the liver at a second time point after an administration of a second contrast agent, wherein healthy liver cells are depicted with contrast enhancement in the second MRI image at the second time point as a consequence of the administration of the first contrast agent, and wherein blood vessels are depicted with contrast enhancement in the second MRI image at the second time point as a consequence of the administration of the second contrast agent.

The control and calculation unit (12) is further configured to generate a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image.

The control and calculation unit (12) is further configured to prompt the output unit (13) to display the first MRI picture, to output it or to store it in a data storage medium.

FIG. 4 shows schematically and exemplarily one embodiment of the method according to the disclosure. The method (100) comprises the steps of:

(110) receiving a first MRI image of an examination object, wherein the first MRI image shows a liver or part of a liver of the examination object at a first time point after an administration of a first contrast agent, wherein healthy liver cells are depicted with contrast enhancement in the first MRI image at the first time point as a consequence of the administration of the first contrast agent,

(120) receiving a second MRI image of the same examination object, wherein the second MRI image shows the same liver or the same part of the liver at a second time point after an administration of a second contrast agent, wherein healthy liver cells are depicted with contrast enhancement in the second MRI image at the second time point as a consequence of the administration of the first contrast agent, and wherein blood vessels are depicted with contrast enhancement in the second MRI image at the second time point as a consequence of the administration of the second contrast agent,

(130) generating a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image,

(140) displaying and/or outputting the first MRI picture and/or storing the first MRI picture in a data storage medium.

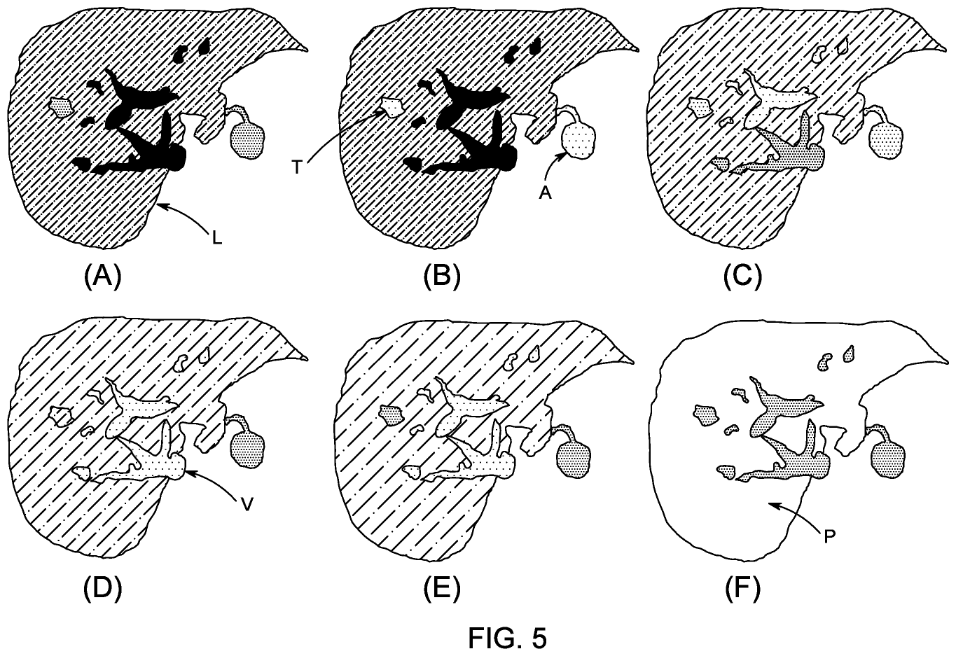

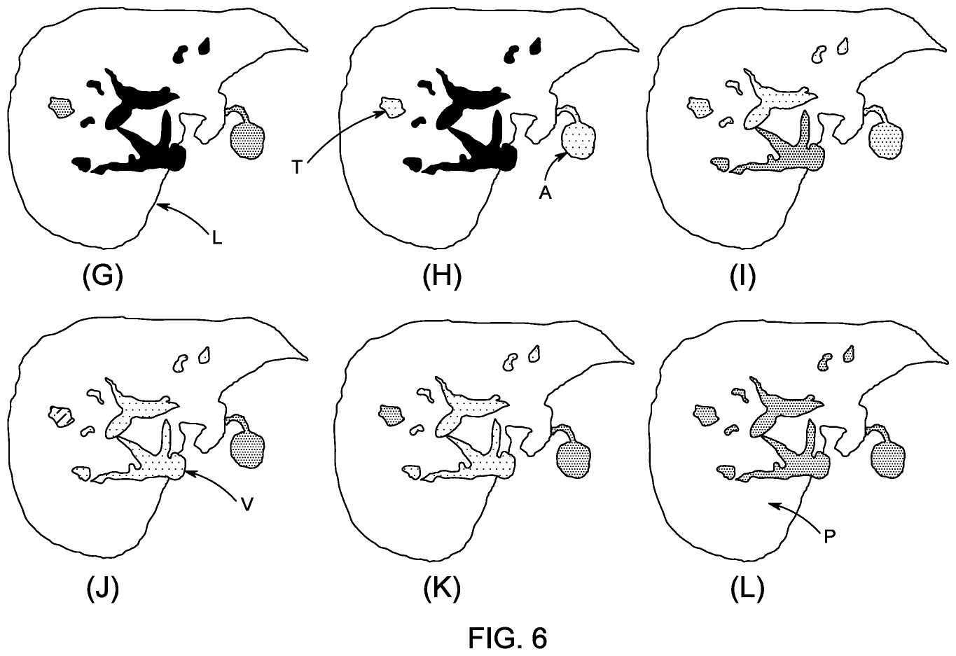

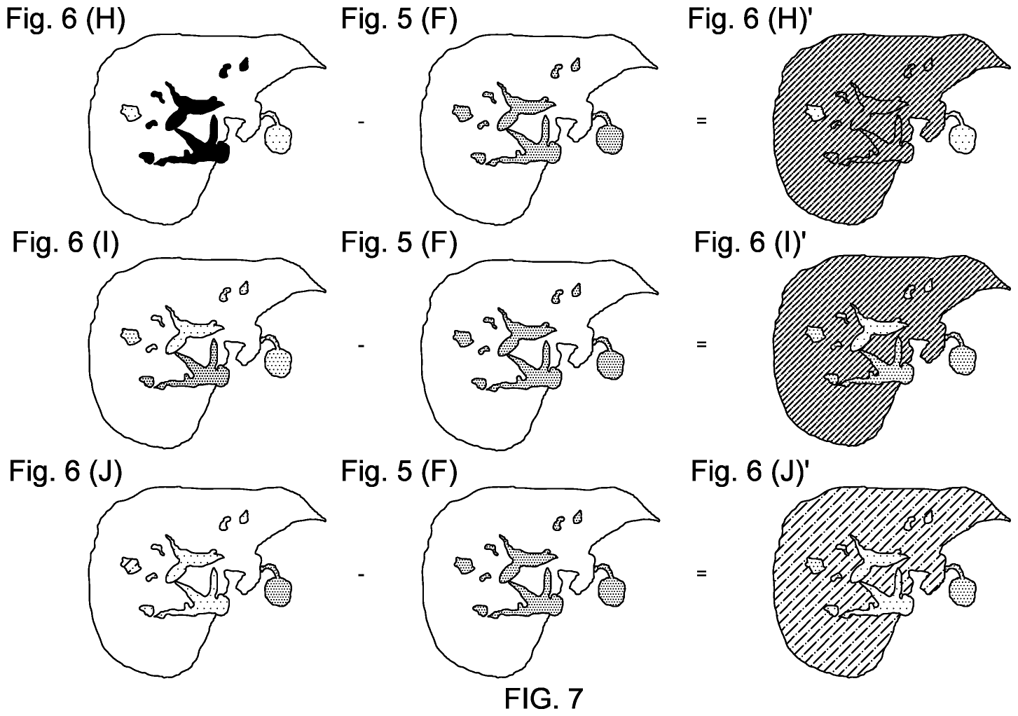

FIG. 5 shows MRI images of a liver during the dynamic and the hepatobiliary phase. In FIGS. 5 (a), 5 (b), 5 (c), 5 (d), 5 (e) and 5 (f), the same cross section through the liver is always depicted at different time points. The reference signs entered in FIGS. 5 (a), 5 (b), 5 (d) and 5 (f) apply to all of FIGS. 5 (a), 5 (b), 5 (c), 5 (d), 5 (e) and 5 (f); they are each entered only once merely for the sake of clarity.

FIG. 5 (a) shows the cross section through the liver (L) before the intravenous administration of a (hepatobiliary) contrast agent. At a time point between the time points depicted by FIGS. 5 (a) and 5 (b), a hepatobiliary contrast agent was administered intravenously as a bolus. This reaches the liver via the liver artery (A) in FIG. 5 (b). Accordingly, the liver artery is depicted with signal enhancement (arterial phase). A tumor (T), which is supplied with blood mainly via arteries, likewise stands out from the liver-cell tissue as a lighter (signal-enhanced) region. At the time point depicted in FIG. 5 (c), the hepatobiliary contrast agent reaches the liver via the veins. In FIG. 5 (d), the venous blood vessels (V) stand out from the liver tissue as light (signal-enhanced) regions (portal venous phase). At the same time, the signal intensity in the healthy liver cells, which are supplied with contrast agent mainly via the veins, continuously rises (FIG. 5 (c)→5 (d)→5 (e)→5 (f)). In the hepatobiliary phase depicted in FIG. 5 (f), the liver cells (P) are depicted with signal enhancement; the blood vessels and the tumor no longer have contrast agent and are accordingly depicted darkly.

According to the disclosure, a first MRI image is generated at a first time point during the hepatobiliary phase. This can, for example, be the image shown in FIG. 5 (f).

FIG. 6 shows the same liver (L) already depicted in FIG. 5. The state shown in FIG. 6 (g) comes after the state shown in FIG. 5 (f). The liver is still depicted with signal enhancement as a consequence of the administration of the hepatobiliary contrast agent. At the time point shown in FIG. 6 (g), contrast agent is administered intravenously a second time in the form of a bolus. The phases already described in connection with FIG. 5 are passed through again: the arterial phase (FIG. 6 (h)), the portal venous phase (FIG. 6 (j)) and the late phase (FIG. 6 (k)). If the second contrast agent is likewise a hepatobiliary contrast agent, a second hepatobiliary phase is also passed through (FIG. 6 (l)), with the difference that the healthy liver cells still contain first contrast agent from the first administration and are depicted with signal enhancement over all phases. Accordingly, the contrast between blood vessels and the healthy liver cells is, especially in the images shown in FIG. 6 (i) and FIG. 6 (j), lower than in the images shown in FIG. 5 (c) and FIG. 5 (d).

To increase this contrast, MRI pictures are generated for one or more of the images shown in FIG. 6 (g), FIG. 6 (h), FIG. 6 (i), FIG. 6 (j) and FIG. 6 (k). This is depicted exemplarily in FIG. 7.

FIG. 7 shows schematically and exemplarily the generation of three MRI pictures 6 (h)′, 6 (i)′ and 6 (j)′. MRI picture 6 (h)′ is generated by subtraction of the MRI image from FIG. 5 (f) from the MRI image from FIG. 6 (h). MRI picture 6 (i)′ is generated by subtraction of the MRI image from FIG. 5 (f) from the MRI image from FIG. 6 (i). MRI picture 6 (j)′ is generated by subtraction of the MRI image from FIG. 5 (f) from the MRI image from FIG. 6 (j). The subtraction is, in each case, done for the tonal values of the individual picture elements of the MRI images. MRI picture 6 (h)′ corresponds to the MRI image shown in FIG. 5 (b); MRI picture 6 (i)′ corresponds to the MRI image shown in FIG. 5 (c); MRI picture 6 (j)′ corresponds to the MRI image shown in FIG. 5 (d).

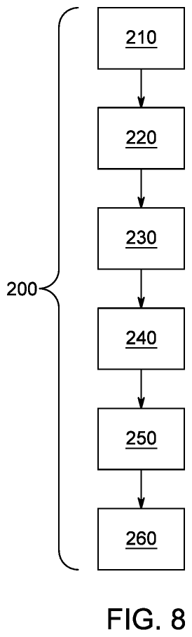

FIG. 8 shows schematically and exemplarily, in the form of a flow chart, a preferred embodiment of the method according to the disclosure. The method (200) comprises the steps of:

(210) receiving a first MRI image of an examination object, wherein the first MRI image shows a liver or part of a liver of the examination object during a first hepatobiliary phase at a first time point after an administration of a first contrast agent,

(220) receiving a second MRI image of the same examination object, wherein the second MRI image shows the same liver or the same part of the liver during a second arterial phase at a second time point after an administration of a second contrast agent,

(230) receiving a third MRI image of the same examination object, wherein the third MRI image shows the same liver or the same part of the liver during a second portal venous phase at a third time point after the administration of the second contrast agent,

(240) receiving a fourth MRI image of the same examination object, wherein the fourth MRI image shows the same liver or the same part of the liver during a second late phase at a fourth time point after the administration of the second contrast agent,

(250) generating MRI pictures from the received MRI images, wherein a first MRI picture is calculated by subtraction of the first MRI image from the second MRI image, wherein a second MRI picture is calculated by subtraction of the first MRI image from the third MRI image, wherein a third MRI picture is calculated by subtraction of the first MRI image from the third MRI image,

(260) displaying and/or outputting one or more of the generated MRI pictures and/or storing one or more of the generated MRI pictures in a data storage medium.

1. A computer-implemented method comprising:
receiving a first MRI image of an examination object, wherein the first MRI image shows a liver or part of a liver of the examination object at a first time point after an administration of a first contrast agent, wherein healthy liver cells are depicted with contrast enhancement in the first MRI image at the first time point as a consequence of the administration of the first contrast agent,
receiving a second MRI image of the same examination object, wherein the second MRI image shows the same liver or the same part of the liver at a second time point after an administration of a second contrast agent, wherein healthy liver cells are depicted with contrast enhancement in the second MRI image at the second time point as a consequence of the administration of the first contrast agent, and wherein blood vessels are depicted with contrast enhancement in the second MRI image at the second time point as a consequence of the administration of the second contrast agent,
generating a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image, and
displaying and/or outputting the first MRI picture and/or storing the first MRI picture in a data storage medium and/or outputting the first MRI picture to a printer.

2. The method of claim 1, wherein the first MRI picture shows the same liver or the same part of the liver after the administration of the second contrast agent, wherein the contrast enhancement of healthy liver cells caused by the first contrast agent has been eliminated.

3. The method of claim 1, further comprising:
receiving the first MRI image of the examination object, wherein the first MRI image shows the liver or the part of a liver of the examination object during a first hepatobiliary phase at the first time point after the administration of the first contrast agent,
receiving the second MRI image of the same examination object, wherein the second MRI image shows the same liver or the same part of the liver during a second arterial phase at the second time point after the administration of the second contrast agent,
optionally receiving a third MRI image of the same examination object, wherein the third MRI image shows the same liver or the same part of the liver during a second portal venous phase at a third time point after the administration of the second contrast agent,
optionally receiving a fourth MRI image of the same examination object, wherein the fourth MRI image shows the same liver or the same part of the liver during a second late phase at a fourth time point after the administration of the second contrast agent,
generating MRI pictures from the received MRI images, wherein the first MRI picture is calculated by subtraction of the first MRI image from the second MRI image, wherein optionally a second MRI picture is calculated by subtraction of the first MRI image from the third MRI image, wherein optionally a third MRI picture is calculated by subtraction of the first MRI image from the third MRI image, and
displaying and/or outputting one or more of the generated MRI pictures and/or storing one or more of the generated MRI pictures in the data storage medium and/or outputting one or more of the generated MRI pictures to a printer.

4. The method of claim 1, further comprising:
feeding the first MRI image and the second MRI image to an artificial neural network, wherein the artificial neural network has been trained in a supervised learning process to generate third reference MRI images from first reference MRI images and second reference MRI images, wherein each first reference MRI image shows a liver or part of a liver of the examination object at a first time point after an administration of a first contrast agent, wherein healthy liver cells are depicted with contrast enhancement in the first reference MRI image at the first time point as a consequence of the administration of the first contrast agent, wherein each second reference MRI image shows the same liver or the same part of the liver at a second time point after an administration of a second contrast agent, wherein healthy liver cells are depicted with contrast enhancement in the second MRI image at the second time point as a consequence of the administration of the first contrast agent and wherein blood vessels are depicted with contrast enhancement in the second MRI image at the second time point as a consequence of the administration of the second contrast agent, and wherein each third reference MRI image shows the same liver or the same part of the liver at the second time point after the administration of the second contrast agent, wherein only blood vessels are depicted with contrast enhancement in the second MRI image at the second time point as a consequence of the administration of the second contrast agent, wherein healthy liver cells are not depicted with contrast enhancement.

5. The method of claim 1, wherein the first contrast agent is a hepatobiliary contrast agent, preferably a substance or a substance mixture having gadoxetic acid or a salt of gadoxetic acid as contrast-enhancing active substance, and the second contrast agent is either the same contrast agent as the first contrast agent or an extracellular contrast agent, preferably a substance or a substance mixture having gadobutrol, gadoteridol, gadoteric acid, gadopentetic acid or gadodiamide as contrast-enhancing active substance.

6. A system comprising:
a receiving unit,
a control and calculation unit and
an output unit,
wherein the control and calculation unit is configured to prompt the receiving unit to receive a first MRI image of an examination object, wherein the first Mill image shows a liver or part of a liver of the examination object at a first time point after an administration of a first contrast agent, wherein healthy liver cells are depicted with contrast enhancement in the first MRI image at the first time point as a consequence of the administration of the first contrast agent,
wherein the control and calculation unit is configured to prompt the receiving unit to receive a second Mill image of an examination object, wherein the second MRI image shows the same liver or the same part of the liver at a second time point after an administration of a second contrast agent, wherein healthy liver cells are depicted with contrast enhancement in the second MRI image at the second time point as a consequence of the administration of the first contrast agent, and wherein blood vessels are depicted with contrast enhancement in the second MRI image at the second time point as a consequence of the administration of the second contrast agent,
wherein the control and calculation unit is configured to generate a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image, and
wherein the control and calculation unit is configured to prompt the output unit to display the first MRI picture, to output it to a printer or to store it in a data storage medium.

7. The computer system of claim 6,
wherein the control and calculation unit is configured to prompt the receiving unit to receive a first MRI image, a second MRI image, a third MRI image and a fourth MRI image,
wherein the first MRI image shows the liver or the part of the liver of the examination object during a first hepatobiliary phase at the first time point after the administration of the first contrast agent,
wherein the second MRI image shows the same liver or the same part of the liver during a second arterial phase at the second time point after the administration of the second contrast agent,
wherein the third MRI image shows the same liver or the same part of the liver during a second portal venous phase at a third time point after the administration of the second contrast agent,
wherein the fourth MRI image shows the same liver or the same part of the liver during a second late phase at a fourth time point after the administration of the second contrast agent,
wherein the control and calculation unit is configured to generate MRI pictures from the received MRI images, wherein a first generated MRI picture is calculated by subtraction of the first MRI image from the second MRI image, wherein a second generated MRI picture is calculated by subtraction of the first MRI image from the third MRI image, wherein a third generated MRI picture is calculated by subtraction of the first MRI image from the third MRI image, and
wherein the control and calculation unit is configured to prompt the output unit to display one or more of the generated MRI pictures and/or to output them to a printer and/or to store them in the data storage medium.

8. A computer program product comprising a computer program which can be loaded into a memory of a computer system, where it prompts the computer system to execute the following:
receiving a first MRI image of an examination object, wherein the first MRI image shows a liver or part of a liver of the examination object at a first time point after an administration of a first contrast agent, wherein healthy liver cells are depicted with contrast enhancement in the first MRI image at the first time point as a consequence of the administration of the first contrast agent,
receiving a second MRI image, wherein the second MRI image shows the same liver or the same part of the liver at a second time point after an administration of a second contrast agent, wherein healthy liver cells are depicted with contrast enhancement in the second MRI image at the second time point as a consequence of the administration of the first contrast agent, and wherein blood vessels are depicted with contrast enhancement in the second MRI image at the second time point as a consequence of the administration of the second contrast agent,
generating a first MRI picture from the first MRI image and the second MRI image, wherein the first MRI picture shows the same liver or the same part of the liver at the second time point, wherein the contrast between healthy liver cells and blood vessels is increased in the first MRI picture compared to the second MRI image, and
displaying the first MRI picture and/or storing the first MRI picture in a data storage medium and/or outputting the first MRI picture to a printer.

9. The computer program product of claim 8, further comprising:
receiving the first MRI image of the examination object, wherein the first MRI image shows the liver or the part of the liver of the examination object during a first hepatobiliary phase at the first time point after the administration of the first contrast agent,
receiving the second MRI image, wherein the second MRI image shows the same liver or the same part of the liver during a second arterial phase at the second time point after the administration of the second contrast agent,
receiving a third MRI image, wherein the third MRI image shows the same liver or the same part of the liver during a second portal venous phase at a third time point after the administration of the second contrast agent,
receiving a fourth MRI image, wherein the fourth MRI image shows the same liver or the same part of the liver during a second late phase at a fourth time point after the administration of the second contrast agent,
generating MRI pictures from the received MRI images, wherein the first generated MRI picture is calculated by subtraction of the first MRI image from the second MRI image, wherein a second generated MRI picture is calculated by subtraction of the first MRI image from the third MRI image, wherein a third generated MRI picture is calculated by subtraction of the first MRI image from the third MRI image, and
displaying one or more of the generated MRI pictures and/or storing one or more of the generated MRI pictures in the data storage medium and/or outputting one or more of the generated MRI pictures to a printer.

10. The computer program product of claim 9, further comprising
feeding the first MRI image and the second MRI image to an artificial neural network, wherein the artificial neural network has been trained in a supervised learning process to generate third reference MRI images from first reference MRI images and second reference MRI images, wherein each first reference MRI image shows a liver or part of a liver of the examination object at a first time point after an administration of a first contrast agent, wherein healthy liver cells are depicted with contrast enhancement in the first reference MRI image at the first time point as a consequence of the administration of the first contrast agent, wherein each second reference MRI image shows the same liver or the same part of the liver at a second time point after an administration of a second contrast agent, wherein healthy liver cells are depicted with contrast enhancement in the second MRI image at the second time point as a consequence of the administration of the first contrast agent and wherein blood vessels are depicted with contrast enhancement in the second MRI image at the second time point as a consequence of the administration of the second contrast agent, and wherein each third reference MRI image shows the same liver or the same part of the liver at the second time point after the administration of the second contrast agent, wherein only blood vessels are depicted with contrast enhancement in the second MRI image at the second time point as a consequence of the administration of the second contrast agent, wherein healthy liver cells are not depicted with contrast enhancement.

11. Use of a first and a second contrast agent in an MRI method, wherein the MRI method comprises:
administering the first contrast agent,
receiving a first MRI image, wherein the first MRI image shows a liver or part of a liver at a first time point after the administration of the first contrast agent, wherein healthy liver cells are depicted with contrast enhancement in the first MRI image at the first time point as a consequence of the administration of the first contrast agent,
administering the second contrast agent at a second time point after the first MRI image is received,
receiving a second MRI image, wherein the second MRI image shows the same liver or the same part of the liver at a second time point after the administration of the second contrast agent, wherein healthy liver cells are depicted with contrast enhancement in the second MRI image at the second time point as a consequence of the administration of the first contrast agent, and wherein blood vessels are depicted with contrast enhancement in the second MRI image at the second time point as a consequence of the administration of the second contrast agent,
generating a first MRI picture from the first MRI image and the second MRI image, wherein the first MRI picture shows the same liver or the same part of the liver at the second time point, wherein the contrast between healthy liver cells and blood vessels is increased in the first Mill picture compared to the second MRI image, and
displaying one or more of the generated MRI pictures and/or storing one or more of the generated MRI pictures in a data storage medium and/or outputting one or more of the generated MRI pictures to a printer.

12. The use of the first and second contrast agent of claim 11, wherein the first MRI image shows the liver or the part of the liver of the examination object during a first hepatobiliary phase at the first time point after the administration of the first contrast agent, wherein the second MRI image shows the same liver or the same part of the liver during a second arterial phase at the second time point after the administration of the second contrast agent, further comprising:
receiving a third MRI image, wherein the third MRI image shows the same liver or the same part of the liver during a second portal venous phase at a third time point after the administration of the second contrast agent,
receiving a fourth MRI image, wherein the fourth MRI image shows the same liver or the same part of the liver during a second late phase at a fourth time point after the administration of the second contrast agent,
generating MRI pictures from the received MRI images, wherein the first MRI picture is calculated by subtraction of the first MRI image from the second MRI image, wherein a second MRI picture is calculated by subtraction of the first MRI image from the third MRI image, wherein a third MRI picture is calculated by subtraction of the first MRI image from the third MRI image, and
displaying one or more of the generated MRI pictures and/or storing one or more of the generated MRI pictures in the data storage medium and/or outputting one or more of the generated MRI pictures to a printer.

13. (canceled)

14. The use of the first and second contrast agent of claim 11, wherein the first contrast agent is a hepatobiliary contrast agent, preferably a substance or a substance mixture having gadoxetic acid or a salt of gadoxetic acid as contrast-enhancing active substance, and the second contrast agent is either the same contrast agent as the first contrast agent or an extracellular contrast agent, preferably a substance or a substance mixture having gadobutrol, gadoteridol, gadoteric acid, gadopentetic acid or gadodiamide as contrast-enhancing active substance.

15. A kit comprising one or more contrast agents according to claim 11 and a computer program product according to claim 8.

16. The use of the first and second contrast agent of claim 12, wherein the first contrast agent is a hepatobiliary contrast agent, preferably a substance or a substance mixture having gadoxetic acid or a salt of gadoxetic acid as contrast-enhancing active substance, and the second contrast agent is either the same contrast agent as the first contrast agent or an extracellular contrast agent, preferably a substance or a substance mixture having gadobutrol, gadoteridol, gadoteric acid, gadopentetic acid or gadodiamide as contrast-enhancing active substance.
